(19) United States
(12) Patent Application Publication
(10) Pub. No.: US 2024/0386996 A1
(43) Pub. Date: Nov. 21, 2024

(54) ARTIFICIAL INTELLIGENCE BASED SYSTEMS AND METHODS FOR ANALYZING MICRO-RIBONUCLEIC ACID (miRNA) SIGNATURE SEQUENCES AND PROFILES IN BIOLOGICAL SAMPLES

(71) Applicant: Convergent Animal Health, LLC, Overland Park, KS (US)

(72) Inventors: Nedal Saleh, San Jose, CA (US); Richard Vierling, Vernon, TX (US)

(21) Appl. No.: 18/671,152

(22) Filed: May 22, 2024

Related U.S. Application Data
(63) Continuation-in-part of application No. 18/320,253, filed on May 19, 2023, now abandoned.

(51) Int. Cl. G16B 20/20; G16B 50/30

(52) U.S. Cl. CPC G16B 20/20; G16B 50/30

(57) ABSTRACT

Artificial intelligence based (AI-based) analyzing Systems and methods for analyzing micro-ribonucleic acid (miRNA) signature profiles in biological sample are disclosed. An artificial intelligence based (AI-based) analyzing system comprises an extractor unit and a nucleic acid amplifying unit that communicate with hardware processors. Extracted miRNA concentrations using the extractor unit are amplified by the nucleic acid amplifying unit, using a plurality of primers, and statistical model-based techniques. The hardware processors analyze miRNA profiling data using statistical model-based techniques with an artificial intelligence model to identify a plurality of miRNA signature sequences and profiles indicative of exposure to biotic and abiotic agents. The processors compare sequences and profiles with pre-defined miRNA signature sequences and profiles in a database and on a display, indicating the subject's response to each biotic or abiotic agent. The artificial intelligence based (AI-based) analyzing system performs multiplexed extraction and amplification of miRNA sequences to improve efficiency.

### CROSS REFERENCE TO RELATED APPLICATION(S)

This application claims the priority to incorporates by reference the entire disclosure of U.S. non-provisional patent application Ser. No. 18/320,253, filed on May 19, 2023, titled “SYSTEMS AND METHODS FOR ANALYZING MICRO-RIBONUCLEIC ACID (miRNA) SIGNATURE PROFILES IN BIOTIC AND ABIOTIC SAMPLES”.

### TECHNICAL FIELD

Embodiments of the present disclosure relate to artificial intelligence based (AI-based) response detection systems and more particularly relate to AI-based analyzing systems and methods for analyzing micro-ribonucleic acid (miRNA) signature sequences and profiles in a biological sample to identify a response of a subject to biotic and abiotic agents.

### BACKGROUND

Generally, animal testing is a common practice where blood, tissue, or bodily fluid samples are collected from either living or dead animals and sent to a testing lab. However, this process can be time-consuming and inefficient, especially if the lab is located in a different facility or location. In addition, testing can be costly if multiple single analyte assays are used. In contrast, multiplex assay capabilities offer several advantages over single detection methods for handling high volumes of samples. These assay panels can be used for early pathogen, biotic and abiotic detection and can significantly improve response time, helping to reduce the impact of infectious disease outbreaks. They can also reveal early signs of onset of a medical condition like abnormal tissue growth.

Further, multiplexing can be achieved through spatial separation, using discrete regions of a channel network, or different detection labels. Current detection technologies include in situ hybridization, quantitative polymerase chain reaction (qPCR), microarrays, next-generation sequencing, lateral flow, electrochemical, and microfluidic chips. Further, micro-ribonucleic acid (miRNA) based assays have proven to be a well-adapted and versatile technology that can be customized to rapidly screen for exposure to DNA and RNA viruses, bacteria, parasites, and prions in a single assay. However, the current detection technologies may not provide rapid and accurate multiplex tests to identify pathogens that have infected an individual.

For example, chronic wasting disease (CWD) is a transmissible spongiform encephalopathy (TSE) that affects the deer family Cervidae, caused by an alternative folding of a prion protein. It results in a continual decline in the animal's health and is fatal. CWD has been documented in both captive and wild mammals, which is transmitted through various routes, including consumption of infected carcasses, antlers, tissues, saliva, feces, urine, blood, placenta, and semen, as well as through contact with contaminated soil, plants, and mineral licks. CWD prions can also be taken up by grasses and sequestered in stems and leaves, potentially serving as a vector for horizontal transfer among herbivore species. Proactive detection methods that are highly sensitive for CWD would allow for critical management policies that minimize the impact of this disease. The currently approved CWD diagnostic assays are limited and invasive, requiring complex procedures and post-mortem tissue sampling.

Hence, there is a need in the art for an artificial intelligence based (AI-based) analyzing system and method for analyzing micro-ribonucleic acid (miRNA) signature sequences and profiles in a biological sample to identify a response of a subject to biotic and abiotic agents, to address at least the aforementioned issues.

### SUMMARY

This summary is provided to introduce a selection of concepts, in a simple manner, which is further described in the detailed description of the disclosure. This summary is neither intended to identify key nor essential inventive concepts of the subject matter nor to determine the scope of the disclosure.

An aspect of the present disclosure provides an artificial intelligence based (AI-based) analyzing system for analyzing micro-ribonucleic acid (miRNA) signature sequences and profiles in a biological sample to identify a response of a subject to biotic and abiotic agents. The artificial intelligence based (AI-based) analyzing system comprises an extractor unit configured to extract each of one or more micro-ribonucleic acid (miRNA) concentrations from one or more candidate pools of the one or more miRNA concentrations in the biological sample. The artificial intelligence based (AI-based) analyzing system further comprises a nucleic acid amplifying unit, communicatively connected to the extractor unit, configured to amplify, using a plurality of primers, each of a plurality of pre-defined nucleic acid sequences and profiles in each of the extracted one or more miRNA concentrations. Further, the artificial intelligence based (AI-based) analyzing system further comprises one or more hardware processors communicatively connected to the nucleic acid amplifying unit; and a memory coupled to the one or more hardware processors. The memory comprises a plurality of modules in form of programmable instructions executable by the one or more hardware processors.

The plurality of modules comprises a profile analyzing module configured to analyze miRNA profiling data using each of the amplified plurality of nucleic acid sequences and profiles from the nucleic acid amplifying unit. The plurality of modules further comprises a signature identifying module configured to identify a plurality of miRNA signature sequences and profiles indicative of an exposure to a plurality of biotic and abiotic agents, based on the analyzed miRNA profiling data. The plurality of miRNA signature sequences is identified using one or more statistical model-based techniques. The one or more statistical model-based techniques comprise artificial intelligence (AI) techniques configured by an artificial intelligence (AI) model.

The plurality of modules further comprises a database comparing module configured to compare each of the plurality of miRNA signature sequences and profiles with each of a plurality of pre-defined miRNA signature sequences and profiles stored in a database. The plurality of modules further comprises a result outputting module configured to output, through a display communicatively connected to the one or more hardware processors, biological status information indicative of a response of a subject to each of the plurality of biotic and abiotic agents, based on a result of the comparison. The plurality of biotic and abiotic agents comprises at least one of: one or more biotic organisms and abiotic stimuli, one or more pathogenesis-related (PR) proteins, one or more antimicrobial peptides (AMPs) induced by phytopathogens, unregulated tissue growth including benign and cancerous tumors, and circulating tumor cells. The one or more biotic organisms comprise at least one of: bacteria, fungi, protozoa, worms, viruses, and parasites. The one or more pathogenesis-related (PR) proteins comprise prions.

Another aspect of the present disclosure provides an artificial intelligence based (AI-based) analyzing method for analyzing micro-ribonucleic acid (miRNA) signature sequences and profiles in a biological sample to identify a response of a subject to biotic and abiotic agents. The artificial intelligence based (AI-based) analyzing method includes extracting each of one or more micro-ribonucleic acid (miRNA) concentrations from one or more candidate pools of the one or more miRNA concentrations in the biological sample. Further, the artificial intelligence based (AI-based) analyzing method includes amplifying using a plurality of primers, each of a plurality of pre-defined nucleic acid sequences and profiles in each of the extracted the one or more miRNA concentrations. Furthermore, the artificial intelligence based (AI-based) analyzing method includes analyzing miRNA profiling data using each of the amplified plurality of nucleic acid sequences and profiles from the nucleic acid amplifying unit. Additionally, the artificial intelligence based (AI-based) analyzing method includes identifying a plurality of miRNA signature sequences and profiles indicative of an exposure to a plurality of biotic and abiotic agents, based on the analyzed miRNA profiling data. The plurality of miRNA signature sequences and profiles is identified using one or more statistical model-based techniques. The one or more statistical model-based techniques comprise artificial intelligence (AI) techniques configured by an artificial intelligence (AI) model. Further, the artificial intelligence based (AI-based) analyzing method includes comparing each of the plurality of miRNA signature sequences and profiles with each of a plurality of pre-defined miRNA signature sequences and profiles stored in a database. Furthermore, the artificial intelligence based (AI-based) analyzing method includes outputting, through a display communicatively connected to the one or more hardware processors, biological status information indicative of a response of a subject to each of the plurality of biotic and abiotic agents, based on a result of the comparison. The plurality of biotic and abiotic agents comprises at least one of: one or more biotic organisms and abiotic stimuli, one or more pathogenesis-related (PR) proteins, one or more antimicrobial peptides (AMPs) induced by phytopathogens, unregulated tissue growth including benign and cancerous tumors, and circulating tumor cells. The one or more biotic organisms comprise at least one of: bacteria, fungi, protozoa, worms, viruses, and parasites. The one or more pathogenesis-related (PR) proteins comprise prions.

Yet another aspect of the present disclosure provides a non-transitory computer-readable storage medium having programmable instructions stored therein. That when executed by one or more hardware processors, causes the one or more hardware processors to analyze miRNA profiling data using each of amplified plurality of nucleic acid sequences and profiles from a nucleic acid amplifying unit. The one or more hardware processors are configured to identify a plurality of miRNA signature sequences and profiles indicative of an exposure to a plurality of biotic and abiotic agents, based on the analyzed miRNA profiling data. The plurality of miRNA signature sequences and profiles is identified using one or more statistical model-based techniques. The one or more statistical model-based techniques comprise artificial intelligence (AI) techniques configured by an artificial intelligence (AI) model. The one or more hardware processors are configured to compare each of the plurality of miRNA signature sequences and profiles with each of a plurality of pre-defined miRNA signature sequences and profiles stored in a database. Further, the one or more hardware processors are configured to output, through a display communicatively connected to the one or more hardware processors, biological status information indicative of a response of a subject to each of the plurality of biotic and abiotic agents, based on a result of the comparison. The plurality of biotic and abiotic agents comprises at least one of: one or more biotic organisms and abiotic stimuli, one or more pathogenesis-related (PR) proteins, one or more antimicrobial peptides (AMPs) induced by phytopathogens, unregulated tissue growth including benign and cancerous tumors, and circulating tumor cells. The one or more biotic organisms comprise at least one of: bacteria, fungi, protozoa, worms, viruses, and parasites. The one or more pathogenesis-related (PR) proteins comprise prions.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF ACCOMPANYING DRAWINGS

The disclosure will be described and explained with additional specificity and detail with the accompanying figures in which:

FIG. 1 illustrates an exemplary block diagram representation of a network architecture implementing an artificial intelligence based (AI-based) analyzing system for analyzing micro-ribonucleic acid (miRNA) signature sequences and profiles in a biological sample to identify a response of a subject to biotic and abiotic agents, in accordance with an embodiment of the present disclosure;

FIG. 2 illustrates an exemplary block diagram representation of the artificial intelligence based (AI-based) analyzing system, such as those shown in FIG. 1, capable of analyzing the micro-ribonucleic acid (miRNA) signature sequences and profiles in the biological sample to identify the response of the subject to the biotic and abiotic agents, in accordance with an embodiment of the present disclosure;

FIG. 3 illustrates an exemplary flow diagram representation of a method for splitting the biological sample into number of initial candidate pool of miRNAs to perform individual extraction and nucleic acid amplification of the miRNAs, according to an example embodiment of the present disclosure;

FIG. 4 illustrates an exemplary flow diagram representation of a method for performing multiplexed extraction and nucleic acid amplification of miRNAs from the biological sample, according to an example embodiment of the present disclosure;

FIG. 5 illustrates an exemplary flow diagram representation of a method for optimizing a set of miRNAs with a sample set of pathogens used in an earlier test for the response of a biotic and abiotic agents in a blind biological sample, according to an example embodiment of the present disclosure;

FIG. 6 illustrates a flow chart depicting an artificial intelligence based (AI-based) analyzing method for analyzing the micro-ribonucleic acid (miRNA) signature sequences and profiles in the biological sample to identify the response of the subject to the biotic and abiotic agents, according to an example embodiment of the present disclosure; and

FIG. 7 illustrates an exemplary block diagram representation of a hardware platform for an implementation of the disclosed artificial intelligence based (AI-based) analyzing system, according to an example embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client, or server computer system) configured by an application may constitute a “module” (or “subsystem”) that is configured and operated to perform certain operations. In one embodiment, the “module” or “subsystem” may be implemented mechanically or electronically, so a module includes dedicated circuitry or logic that is permanently configured (within a special-purpose processor) to perform certain operations. In another embodiment, a “module” or s “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired), or temporarily configured (programmed) to operate in a certain manner and/or to perform certain operations described herein.

Embodiments of the present disclosure provide an artificial intelligence based (AI-based) analyzing system and an artificial intelligence based (AI-based) analyzing method for analyzing micro-ribonucleic acid (miRNA) signature sequences in a biological sample to identify a response of a subject to biotic and abiotic agents. The present disclosure provides a comprehensive ecosystem comprising multiplexed miRNA assay, novel microfluidic device, portable analysis station, differential expression predictive statistical modeling techniques, and a scalable, multi-pathogen database, which can be monetized. The present disclosure provides the artificial intelligence based (AI-based) analyzing system and method for detecting exposure to a wide range of pathogens, including DNA and RNA viruses, bacteria, fungi, and prions, in a single assay. The present disclosure provides the artificial intelligence based (AI-based) analyzing system and method for measuring changes in the regulatory elements that control transcription and translation, specifically miRNA, to identify the exposure to multiple pathogens, which can be both qualitative and quantitative. The present disclosure provides the artificial intelligence based (AI-based) analyzing system and method for identifying multiple miRNA signatures indicating the exposure to multiple pathogens in a single assay, based on a scalable and a monetizable multi-disease database. The present disclosure employs statistical model-based techniques that compares the results from the models of the statistical model-based techniques to a dynamic database to improve the predictive model. The present disclosure may provide a portable apparatus that can detect a subject response to at least two pathogens and the response of circulating and non-circulating miRNAs using a set of oligonucleotides for the signature of any miRNA.

The present disclosure provides a dynamic database that is monetizable through direct fiat exchange or by fungible and non-fungible tokenization. The miRNA assay offers several advantages over earlier tests for high throughput chronic wasting disease (CWD) testing. Firstly, it provides complementary results to immunohistochemical (IHC), thereby serving as an early detection tool that would provide valuable information for surveillance and identification of asymptomatic animals. Secondly, the ease and speed at which miRNA assays can be completed allow for a sample to pass through the entire process (sample processing and RNA isolation to data processing) in under for e.g., 12 hours, making it a more efficient testing method. The miRNA assay also has several potential applications, including the development of an antemortem blood assay that could prove useful for early detection/rapid response frameworks for CWD surveillance. Ante-mortem sampling using miRNA testing allows for a high throughput capacity and rapid return of results (same day for a single sample assay, in most cases), repeated testing using the same individual with minimal handling and stress to animals, and selective removal of known positive animals. The miRNA assay also allows for continual monitoring or periodic testing in geographic locations or facilities that report positive individuals, enabling landowners, deer breeders, state, and federal agencies to develop methods for management and surveillance for CWD in deer herds. It also serves as an early detection method (compared to IHC) for asymptomatic individuals and establishes a scientific method to determine an elapsed timeframe from exposure to manifestation of CWD symptoms and death. This provides the tools necessary to track disease progression in captive herds and define miRNA changes indicative of different stages of infection (early, onset, late), potentially avoiding circumstances that might lead to depopulation of entire herds.

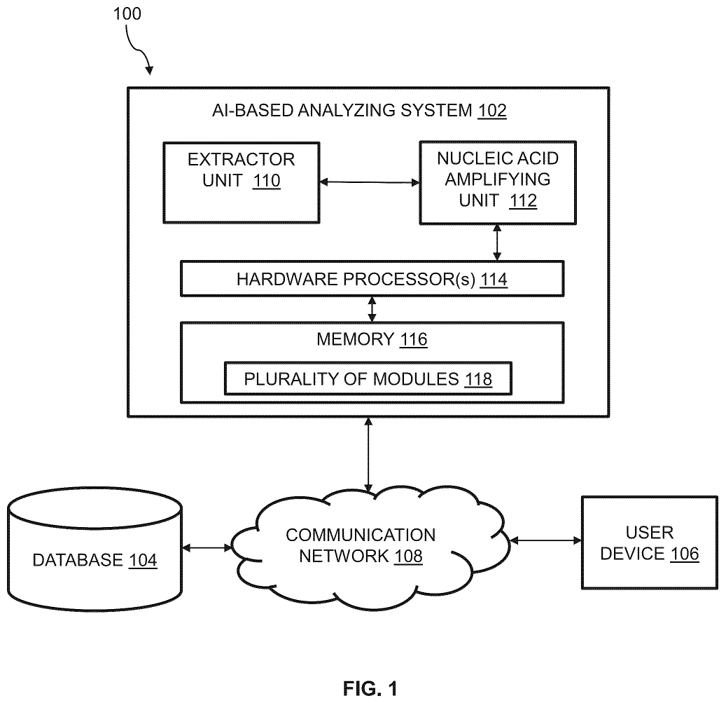

Referring now to the drawings, and more particularly to FIG. 1 through FIG. 7, where similar reference characters denote corresponding features consistently throughout the figures, there are shown preferred embodiments and these embodiments are described in the context of the following exemplary system and/or method.

FIG. 1 illustrates an exemplary block diagram representation of a network architecture 100 implementing an artificial intelligence based (AI-based) analyzing system 102 for analyzing micro-ribonucleic acid (miRNA) signature sequences and profiles in a biological sample to identify a response of a subject to biotic and abiotic agents, in accordance with an embodiment of the present disclosure. According to FIG. 1, the network architecture 100 may include the artificial intelligence based (AI-based) analyzing system 102, a database 104, and a user device 106. The artificial intelligence based (AI-based) analyzing system 102 may be communicatively coupled to the database 104, and the user device 106 via a communication network 108. The communication network 108 may be a wired communication network and/or a wireless communication network. The database 104 may include, but is not limited to, micro-ribonucleic acid (miRNA) concentrations, miRNA signature sequences, miRNA profiling data, a miRNA response, a miRNA absence, miRNA quantitative levels, miRNA concentrations, miRNA expression patterns, miRNA relationships within the miRNA profiling data, any other content, and combinations thereof. The database 104 may be any kind of database such as, but are not limited to, relational databases, dynamic databases, monetized databases, scalable databases, miRNA databases, cloud databases, distributed databases, any other databases, and combination thereof.

The micro-ribonucleic acid (miRNA) is a type of single-stranded non-coding RNA molecule typically consisting of about 20 nucleotides, in bodily tissues or fluids including blood, urine, saliva, eye and nasal secretions, semen, lymph, milk, anal and vaginal secretions, feces, muscle, and organs of a subject. The present disclosure uses miRNA as biomarkers. The miRNA may be circulating in the blood or lymph system, or non-circulating and residing in bodily tissues, fluids, or organs. The subject may be an animal, and the like. The artificial intelligence based (AI-based) analyzing system 102 amplifies the miRNAs using cyclic or isothermal polymerase enzymes, followed by a computational screening and identification to determine the response, absence, and/or quantitative levels of miRNA in absolute or relative measures. The results are then compared to a multi-disease database to identify abnormal protein structures, pathogens, and parasites, collectively referred to as pathogens, to provide rapid, sensitive, specific, and accurate tests for detecting pathogens. The present disclosure also provides miRNA signatures in response to biotic and abiotic organisms or proteins, and the direct detection of miRNA indicative of the response of a foreign pathogen. Furthermore, the present disclosure provides primers capable of amplifying miRNA and computational methods for developing miRNA signatures.

Further, the user device 106 may be associated with, but not limited to, a user, an individual, an administrator, a vendor, a technician, a health care worker, a caretaker, a patient, a supervisor, a team, an entity, a facility, and the like. The user device 106 may be used to provide input and/or receive output to/from the system 102, and/or to the database 104. The user device 106 may present to the user one or more user interfaces for the user to interact with the artificial intelligence based (AI-based) analyzing system 102 and/or to the database 104 for the micro-ribonucleic acid (miRNA) analyzing needs. The user device 106 may be at least one of, an electrical, an electronic, an electromechanical, and a computing device. The user device 106 may include, but is not limited to, a mobile device, a smartphone, a Personal Digital Assistant (PDA), a tablet computer, a phablet computer, a wearable computing device, a Virtual Reality/Augmented Reality (VR/AR) device, a laptop, a desktop, a server, and the like. The entities and the facility may include, but are not limited to, a hospital, a healthcare facility, a laboratory facility, an e-commerce company, a merchant organization, an airline company, a hotel booking company, a company, an outlet, a manufacturing unit, an enterprise, an organization, an educational institution, a secured facility, a warehouse facility, a supply chain facility, any other facility and the like.

Further, the artificial intelligence based (AI-based) analyzing system 102 may be implemented by way of a single device or a combination of multiple devices that may be operatively connected or networked together. The artificial intelligence based (AI-based) analyzing system 102 may be implemented in hardware or a suitable combination of hardware and software. The artificial intelligence based (AI-based) analyzing system 102 includes an extractor unit 110, a nucleic acid amplifying unit 112, one or more hardware processor(s) 114 and a memory 116. The memory 116 may include a plurality of modules 118. The artificial intelligence based (AI-based) analyzing system 102 may be a hardware device including the hardware processor 114 executing machine-readable program instructions for analyzing micro-ribonucleic acid (miRNA) signature sequences and profiles in a biological sample to identify a response of a subject to biotic and abiotic agents. Execution of the machine-readable program instructions by the hardware processor 114 may enable the proposed artificial intelligence based (AI-based) analyzing system 102 to analyze micro-ribonucleic acid (miRNA) signature sequences in a biological sample for identifying a response of a subject to biotic and abiotic agents. The “hardware” may comprise a combination of discrete components, an integrated circuit, an application-specific integrated circuit, a field-programmable gate array, a digital signal processor, or other suitable hardware. The “software” may comprise one or more objects, agents, threads, lines of code, subroutines, separate software applications, two or more lines of code, or other suitable software structures operating in one or more software applications or on one or more processors.

The hardware processor 114 may include, for example, microprocessors, microcomputers, microcontrollers, digital signal processors, central processing units, state machines, logic circuits, and/or any devices that manipulate data or signals based on operational instructions. Among other capabilities, the one or more hardware processor 114 may fetch and execute computer-readable instructions in a memory operationally coupled with the system 102 for performing tasks such as data processing, input/output processing, and/or any other functions. Any reference to a task in the present disclosure may refer to an operation being or that may be performed on data.

Though few components and subsystems are disclosed in FIG. 1, there may be additional components and subsystems which is not shown, such as, but not limited to, assets, machinery, instruments, facility equipment, life safety devices, intensive care devices, treatment devices, emergency management devices, health care devices, laboratory devices, testing kits, any other devices, and combination thereof. The person skilled in the art should not be limiting the components/subsystems shown in FIG. 1. Although FIG. 1 illustrates the artificial intelligence based (AI-based) analyzing system 102, and the user device 106 connected to the database 104, one skilled in the art can envision that the artificial intelligence based (AI-based) analyzing system 102, and the user device 106 can be connected to several user devices located at different locations and several databases via the communication network 108.

Those of ordinary skilled in the art will appreciate that the hardware depicted in FIG. 1 may vary for particular implementations. For example, other peripheral devices such as an optical disk drive and the like, local area network (LAN), wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the system 102 as is unique to the present disclosure or necessary for an understanding of the present disclosure is depicted and described. The remainder of the construction and operation of the system 102 may conform to any of the various current implementations and practices that were known in the art.

In an exemplary embodiment, the artificial intelligence based (AI-based) analyzing system 102 may execute the extractor unit 110 to extract each of one or more micro-ribonucleic acid (miRNA) concentrations from one or more candidate pools of the one or more miRNA concentrations in the biological sample. In an exemplary embodiment the biological sample may be extracted from a subject. The subject may include, but are not limited to, an animal, and the like. The biological sample may be extracted from a bodily tissue or a fluid such as blood, urine, saliva, eye and nasal secretions, semen, lymph, milk, anal secretions, vaginal secretions, feces, muscle, organs, any other parts of animal, and/or combination thereof.

In an exemplary embodiment, the artificial intelligence based (AI-based) analyzing system 102 may execute the nucleic acid amplifying unit 112 communicatively connected to the extractor unit 110. The nucleic acid amplifying unit 112 may be configured to amplify, using a plurality of primers, each of a plurality of pre-defined nucleic acid sequences in each of the extracted the one or more miRNA concentrations. In an exemplary embodiment, amplifying each of the plurality of pre-defined nucleic acid sequences is based on at least one of thermal-cyclic polymerase enzymes and isothermal polymerase enzymes. The Nucleic acid amplification refers to a process of generating multiple copies of a specific nucleic acid sequence, such as a deoxyribonucleic acid DNA or a Ribonucleic acid (RNA), using enzymatic reactions. This technique is widely used in various fields of biology and medicine, such as molecular biology, genetic testing, and disease diagnosis. There are several methods of nucleic acid amplification, including polymerase chain reaction (PCR), loop-mediated isothermal amplification (LAMP), and transcription-mediated amplification (TMA). These methods vary in their efficiency, speed, and technical requirements, but they all involve the use of specific enzymes and primers to selectively amplify the target sequence.

In an exemplary embodiment, the artificial intelligence based (AI-based) analyzing system 102 may execute the one or more hardware processors 114 communicatively connected to the nucleic acid amplifying unit 112. The one or more hardware processors may be configured to analyze miRNA profiling data using each of the amplified plurality of nucleic acid sequences and profiles from the nucleic acid amplifying unit 112. The miRNA profiling data comprises, but not limited to, a miRNA response, a miRNA absence, miRNA quantitative levels, miRNA concentrations, miRNA expression patterns, miRNA relationships within the miRNA profiling data, and the like.

In an exemplary embodiment, the artificial intelligence based (AI-based) analyzing system 102 may execute the one or more hardware processors 114 to identify a plurality of miRNA signature sequences and profiles indicative of an exposure to a plurality of biotic and abiotic agents, based on the analyzed miRNA profiling data. The plurality of miRNA signature sequences and profiles is identified using one or more statistical model-based techniques. The plurality of biotic and abiotic agents comprises at least one of, but not limited to, one or more biotic and abiotic organisms, one or more pathogenesis-related (PR) proteins, and the like. The one or more biotic and abiotic organisms comprise at least one of, but not limited to, bacteria, fungi, protozoa, worms, viruses, parasites, and the like. The one or more pathogenesis-related (PR) proteins comprise prions, and the like. The one or more statistical model-based techniques include, but are not limited to, regression analyses techniques, pair-wise regression analyses techniques, linear regression analyses techniques, multivariate analyses techniques, artificial intelligence (AI) techniques, machine learning (ML) techniques, any other statistical model-based techniques, and a combination thereof.

In an exemplary embodiment, the artificial intelligence based (AI-based) analyzing system 102 may execute the one or more hardware processors 114 to compare each of the plurality of miRNA signature sequences and profiles with each of a plurality of pre-defined miRNA signature sequences and profiles stored in the database 104.

In an exemplary embodiment, the artificial intelligence based (AI-based) analyzing system 102 may execute the one or more hardware processors 114 to output, through a display communicatively connected to the one or more hardware processors 114, biological status information indicative of a response of a subject to each of the plurality of biotic and abiotic agents, based on a result of the comparison. The biological status information refers to any information related to the health or functioning of the subject.

In an exemplary embodiment, the artificial intelligence based (AI-based) analyzing system 102 analyzes micro-ribonucleic acid (miRNA) signature sequences in a biological sample, by multiplexed extraction and multiplexed amplification of micro-ribonucleic acid (miRNA) concentrations. In an exemplary embodiment, the extractor unit 110 may be configured for multiplexed extraction of a plurality of micro-ribonucleic acid (miRNA) concentrations from one or more candidate pools of the plurality of miRNA concentrations in the biological sample. In an exemplary embodiment, the nucleic acid amplifying unit 112 communicatively connected to the extractor unit 110, may be configured for multiplexed amplification, using a plurality of primers, of the plurality of pre-defined nucleic acid sequences in the multiplexed extraction of the plurality of miRNA concentrations. Further, the one or more hardware processor 114 may be configured to analyze the miRNA profiling data using the multiplexed amplification of the plurality of nucleic acid sequences and profiles from the nucleic acid amplifying unit 112.

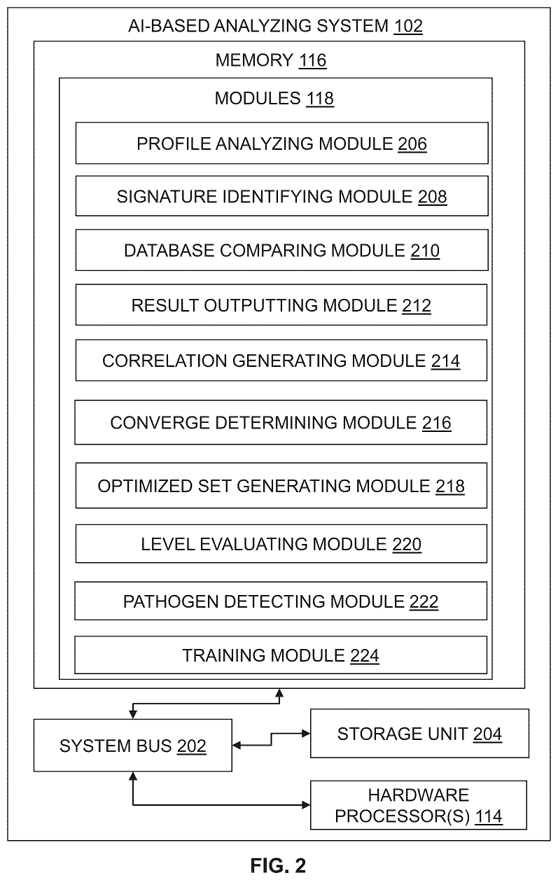

FIG. 2 illustrates an exemplary block diagram representation of the artificial intelligence based (AI-based) analyzing system 102, such as those shown in FIG. 1, capable of analyzing the micro-ribonucleic acid (miRNA) signature sequences and profiles in the biological sample to identify the response of the subject to the biotic and abiotic agents, in accordance with an embodiment of the present disclosure. The artificial intelligence based (AI-based) analyzing system 102 may also function as a computer-implemented system (hereinafter referred to as the system 102). The artificial intelligence based (AI-based) analyzing system 102 comprises the one or more hardware processors 114, the memory 116, and a storage unit 204. The one or more hardware processors 114, the memory 116, and the storage unit 204 are communicatively coupled through a system bus 202 or any similar mechanism. The memory 116 comprises a plurality of modules 118 in the form of programmable instructions executable by the one or more hardware processors 114.

Further, the plurality of modules 118 includes a profile analyzing module 206, a signature identifying module 208, a database comparing module 210, a result outputting module 212, a correlation generating module 214, a converge determining module 216, an optimized set generating module 218, a level evaluating module 220, a pathogen detecting module 222, and a training module 224.

The one or more hardware processors 114, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 114 may also include embedded controllers, such as generic or programmable logic devices or arrays, application-specific integrated circuits, single-chip computers, and the like.

The memory 116 may be a non-transitory volatile memory and a non-volatile memory. The memory 116 may be coupled to communicate with the one or more hardware processors 114, such as being a computer-readable storage medium. The one or more hardware processors 114 may execute machine-readable instructions and/or source code stored in the memory 116. A variety of machine-readable instructions may be stored in and accessed from the memory 116. The memory 116 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory 116 includes the plurality of modules 118 stored in the form of machine-readable instructions on any of the above-mentioned storage media and may be in communication with and executed by the one or more hardware processors 114.

The storage unit 204 may be a cloud storage or a database such as those shown in FIG. 1. The storage unit 204 may store micro-ribonucleic acid (miRNA) concentrations, miRNA signature sequences, miRNA profiling data, a miRNA response, a miRNA absence, miRNA quantitative levels, miRNA concentrations, miRNA expression patterns, miRNA relationships within the miRNA profiling data, any other content, and combinations thereof. The storage unit 204 may be any kind of database such as, but are not limited to, relational databases, dynamic databases, monetized databases, scalable databases, 18iRbase databases, cloud databases, distributed databases, any other databases, and combination thereof.

In an exemplary embodiment, the artificial intelligence based (AI-based) analyzing system 102 may execute the extractor unit 110 to extract each of one or more micro-ribonucleic acid (miRNA) concentrations from the one or more candidate pools of the one or more miRNA concentrations in the biological sample. In an exemplary embodiment the biological sample may be extracted from the subject. The subject may include, but are not limited to, an animal, and the like. The biological sample may be extracted from a bodily tissue or a fluid such as blood, urine, saliva, eye and nasal secretions, semen, lymph, milk, anal secretions, vaginal secretions, feces, muscle, organs, any other parts of animal, and/or combination thereof.

In an exemplary embodiment, the artificial intelligence based (AI-based) analyzing system 102 may execute the nucleic acid amplifying unit 112 communicatively connected to the extractor unit 110. The nucleic acid amplifying unit 112 may be configured to amplify, using the plurality of primers, each of the plurality of pre-defined nucleic acid sequences in each of the extracted the one or more miRNA concentrations. In an exemplary embodiment, amplifying each of the plurality of pre-defined nucleic acid sequences is based on at least one of thermal-cyclic polymerase enzymes and isothermal polymerase enzymes.

In an exemplary embodiment, the profile analyzing module 206 may analyze the miRNA profiling data using each of the amplified plurality of nucleic acid sequences and profiles from the nucleic acid amplifying unit 112. The miRNA profiling data comprises, but not limited to, a miRNA response, a miRNA absence, miRNA quantitative levels, miRNA concentrations, miRNA expression patterns, miRNA relationships within the miRNA profiling data, and the like.

In an exemplary embodiment, the signature identifying module 208 may identify a plurality of miRNA signature sequences and profiles indicative of the exposure to the plurality of biotic and abiotic agents, based on the analyzed miRNA profiling data, such exposure may be presymptomatic. To identify the plurality of miRNA signature sequences and profiles, the signature identifying module 208 may further determine one or more differentially expressed miRNA concentrations corresponding to at least one of biological contexts and biological conditions, using the one or more statistical model-based techniques. To identify the plurality of miRNA signature sequences and profiles, the signature identifying module 208 may further determine one or more miRNA expression patterns associated with at least one of biological processes and disease states, using the one or more statistical model-based techniques.

The plurality of miRNA signature sequences is identified using one or more statistical model-based techniques. The plurality of biotic and abiotic agents comprises at least one of, but not limited to, one or more biotic and abiotic organisms, one or more pathogenesis-related (PR) proteins, one or more antimicrobial peptides (AMPs) induced by phytopathogens, and the like. The one or more biotic and abiotic organisms comprise at least one of, but not limited to, bacteria, fungi, protozoa, worms, viruses, parasites, and the like. The one or more pathogenesis-related (PR) proteins comprise prions, and the like. The one or more statistical model-based techniques include, but are not limited to, regression analyses techniques, pair-wise regression analyses techniques, linear regression analyses techniques, multivariate analyses techniques, artificial intelligence (AI) techniques, machine learning (ML) techniques, any other statistical model-based techniques, and a combination thereof. In an embodiment, the one or more statistical model-based techniques may include the artificial intelligence (AI) techniques configured by an artificial intelligence (AI) model.

In an example, the system 102 may elucidate miRNA signatures by identifying and characterizing the patterns of expression of miRNAs in a particular biological sample. A miRNA signature can be defined as a set of miRNAs whose expression levels are consistently altered in a specific condition or disease, compared to a reference state or control. Elucidating miRNA signatures involves using various molecular biology techniques, such as miRNA sequencing, microarray analysis, or quantitative polymerase chain reaction (qPCR), to measure the expression levels of miRNAs in a sample. By comparing the expression levels of miRNAs in a diseased sample to those in a healthy control, researchers can identify miRNAs that are upregulated or downregulated in the disease state, and thus may be involved in the disease pathogenesis. Further, elucidating miRNA signatures can have important diagnostic, prognostic, and therapeutic implications. For example, miRNA signatures have been identified in various diseases, including cancer, cardiovascular disease, and neurological disorders, and can be used as biomarkers for disease diagnosis or prognosis. In addition, by targeting specific miRNAs that are dysregulated in a disease, researchers can develop miRNA-based therapies to treat the disease.

In an exemplary embodiment, the database comparing module 210 may compare each of the plurality of miRNA signature sequences and profiles with each of a plurality of pre-defined miRNA signature sequences and profiles stored in the database 104.

In an exemplary embodiment, the result outputting module 212 may output, through a display communicatively connected to the one or more hardware processors 114, the biological status information indicative of the response of the subject to each of the plurality of biotic and abiotic agents, based on the result of the comparison.

In an exemplary embodiment, the correlation generating module 214 may generate a multidimensional correlation matrix corresponding to the plurality of miRNA signature sequences with the plurality of biotic and abiotic agents using the one or more statistical model-based techniques. The converge determining module 216 may determine a convergence of the plurality of miRNA signature sequences into a pathway corresponding to the plurality of biotic and abiotic agents, based on the generated multidimensional correlation matrix. For example, MiRNA convergence refers to the phenomenon where multiple miRNAs converge on a single target gene or pathway, resulting in a coordinated regulation of gene expression. The MiRNA convergence is a complex process that involves multiple layers of regulation. In some cases, multiple miRNAs may target different sites within the same mRNA molecule, leading to synergistic effects on gene expression. Alternatively, miRNAs may target different genes within the same pathway, resulting in a cumulative effect on pathway regulation. The phenomenon of miRNA convergence has important implications for understanding the regulatory elements (miRNA) that control gene expression. By targeting multiple genes within a pathway, miRNAs can fine-tune the expression of specific genes and modulate cellular responses to various stimuli. This level of control is particularly important in complex biological processes such as development, differentiation, and disease progression.

Further, the optimized set generating module 218 may generate an optimized subset from the plurality of miRNA signature sequences and profiles, when a first subset of the plurality of miRNA signature sequences and profiles is determined to be converged into the pathway. Furthermore, the correlation generating module 214 may generate a second subset multidimensional correlation matrix corresponding to a second subset of the plurality of miRNA signature sequences and profiles, when a second subset of the plurality of miRNA signature sequences and profiles is determined to be not converged into the pathway. Additionally, the database comparing module 210 may compare each of the first subset and the second subset of the plurality of miRNA signature sequences and profiles with each of a plurality of pre-defined miRNA signature sequences and profiles stored in the database. Further, the level evaluating module 220 may evaluate a statistical confidence level of the multidimensional correlation matrix and the second subset multidimensional correlation matrix, based on a result of the comparison. Furthermore, the pathogen detecting module 222 may detect a response of a pathogen in a blind biological sample using the first subset of the plurality of miRNA signature sequences and profiles, based on a result of the evaluation.

In an alternative embodiment, for identifying, by the artificial intelligence (AI) model, the plurality of miRNA signature sequences and profiles, based on the analyzed miRNA profiling data, the signature identifying module 208 is configured to obtain the miRNA profiling data from the profile analyzing module 206. The signature identifying module 208 is further configured to process the miRNA profiling data by at least one of: removing one or more outliers associated with the miRNA profiling data, normalizing one or more expression values associated with the miRNA profiling data, and handling one or more missing values associated with the miRNA profiling data.

The signature identifying module 208 is further configured to extract one or more features from the miRNA profiling data. The extracted one or more features may include at least one of: one or more expression levels of the miRNAs, one or more statistical measures including mean and standard values, and one or more information including at least one of: target gene information and pathway enrichment, derived from one or more biological insights. The signature identifying module 208 is further configured to identify the plurality of miRNA signature sequences and profiles indicative of the exposure to the plurality of biotic and abiotic agents cause change, based on feature space associated with the miRNA profiling data, using the artificial intelligence (AI) model.

In an exemplary embodiment, the training module 224 is configured to train the artificial intelligence (AI) model. For training the artificial intelligence (AI) model, the training module 224 is configured to obtain one or more training datasets associated with the analyzed miRNA profiling data. The training module 224 is further configured to train the artificial intelligence (AI) model on the one or more training datasets to identify the plurality of miRNA signature sequences and profiles. In an embodiment, the one or more training datasets may include at least one of: one or more proprietary data generated in the workflow and possibly combined with other information corresponding to the analyzed miRNA profiling data.

The training module 224 is further configured to validate performance of the artificial intelligence (AI) model on one or more validation datasets. The training module 224 is further configured to update one or more hyperparameters associated with the analyzed miRNA profiling data to optimize the performance of the artificial intelligence (AI) model in identifying the plurality of miRNA signature sequences and profiles. The training module 224 is further configured to evaluate the trained artificial intelligence (AI) model using one or more metrics including at least one of: accuracy metric, precision metric, recall metric, F1-score metric, Area Under the Precision Recall Curve (AUPRC), Receiver operating characteristic (ROC) curves, Multi-Class Confusion Matrices, and the like.

In an exemplary embodiment, the artificial intelligence based (AI-based) analyzing system 102 may analyze the micro-ribonucleic acid (miRNA) signature sequences and profiles in the biological sample, by multiplexed extraction and multiplexed amplification of the micro-ribonucleic acid (miRNA) concentrations. In an exemplary embodiment, the extractor unit 110 may be configured for multiplexed extraction of the plurality of micro-ribonucleic acid (miRNA) concentrations from the one or more candidate pools of the plurality of miRNA concentrations in the biological sample. In an exemplary embodiment, the nucleic acid amplifying unit 112 communicatively connected to the extractor unit 110, may be configured for multiplexed amplification, using the plurality of primers, of the plurality of pre-defined nucleic acid sequences in the multiplexed extraction of the plurality of miRNA concentrations. Further, the profile analyzing module 206 may analyze the miRNA profiling data using the multiplexed amplification of the plurality of nucleic acid sequences and profiles from the nucleic acid amplifying unit 112.

In addition, the storage unit 204 may be configured to receive from a user associated with the user device 106, a request to update data. The storage unit 204 may provide access, in response to the received request, to at least one of data, personal diagnostics data, and serological profile data, for real-time estimation of an immunological gap. Further, the storage unit 204 may receive from the user associated with the user device 106, at least one of the pre-defined miRNA signature sequences corresponding to the plurality of biotic and abiotic agents, and miRNA data. Furthermore, the storage unit 204 may convert the at least one of the pre-defined miRNA signature sequences corresponding the plurality of biotic and abiotic agents, and miRNA data into at least one of a digital asset, a fungible token, and non-fungible tokenization to provide one or more services to the user. The storage unit 204 may be deployed on at least one of: centralized and decentralized architecture like blockchain.

The one or more services include, but are not limited to incentivization service, trading of the tokens on primary or secondary markets, allowing members of the public to invest in or trade the database. The primary markets refer to the initial sale of the tokens by the issuer, while secondary markets refer to the subsequent trading of the tokens among investors. This means that anyone can participate in buying or selling the tokens, as long as they have access to the relevant marketplaces. Tokenizing a database and making it open to public participation in primary or secondary markets can provide a new way to finance and manage databases, as well as potentially increase their liquidity and value. Further, the digital asset may be exchanged for traditional currency, directly without the need for an intermediary cryptocurrency. For example, if a company creates a digital token that represents ownership in a specific asset or product, they may allow investors to purchase the token directly with fiat currency, rather than requiring them to first convert the fiat currency into a cryptocurrency like Bitcoin or Ethereum.

Furthermore, the storage unit 204 may compile the miRNA profiling data corresponding to the miRNA signature sequences and profiles, miRNA data, as an electronic file. Additionally, the storage unit 204 may receive a request from the user to compare the plurality of miRNA signature sequences and profiles with the compiled miRNA profiling data. Further, the storage unit 204 may generate actionable data and results of the comparison, using one or more statistical model-based techniques. The actionable data comprises, but not limited to, deploying vaccine substances, therapeutics, preventive measures, and the like. To deploy vaccine substances to various elements of the environment, such as animals, water, soil, plants, and other environmental elements. The preventive measures help in identifying the areas or populations that are more susceptible to a particular disease or virus, and thus guide the deployment of vaccines to those areas to prevent the spread of the disease.

Further, the storage unit 204 may store the actionable data and the results of the comparison for analysis, subsequent comparison, and training of the statistical model-based techniques.

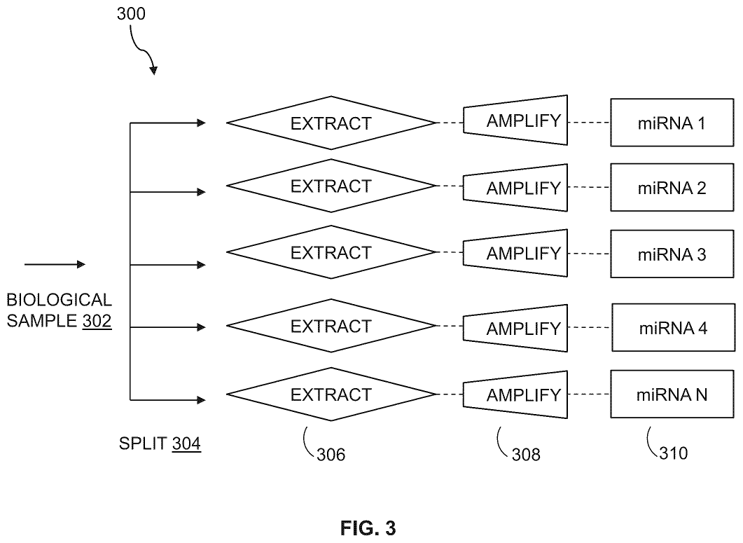

FIG. 3 illustrates an exemplary flow diagram representation of a method 300 (e.g., the artificial intelligence based (AI-based) analyzing method) for splitting the biological sample into number of first candidate pool of miRNAs to perform individual extraction and nucleic acid amplification of the miRNAs, according to an example embodiment of the present disclosure.

At step 302, the method 300 includes, receiving, by the artificial intelligence based (AI-based) analyzing system 102, the biological sample. At step 304, the method 300 includes, splitting, by the artificial intelligence based (AI-based) analyzing system 102, the biological sample into a pool of candidates. At step 306, the method 300 includes extracting each of the one or more micro-ribonucleic acid (miRNA) concentrations from the one or more candidate pools of the one or more miRNA concentrations in the biological sample. At step 308, the method 300 includes amplifying, by the artificial intelligence based (AI-based) analyzing system 102, using the plurality of primers, each of the plurality of pre-defined nucleic acid sequences in each of the extracted the one or more miRNA concentrations. At step 310, the method 300 includes, identifying, by the artificial intelligence based (AI-based) analyzing system 102, the plurality of miRNA signature sequences and profiles indicative of the exposure to the plurality of biotic and abiotic agents, based on the miRNA profiling data.

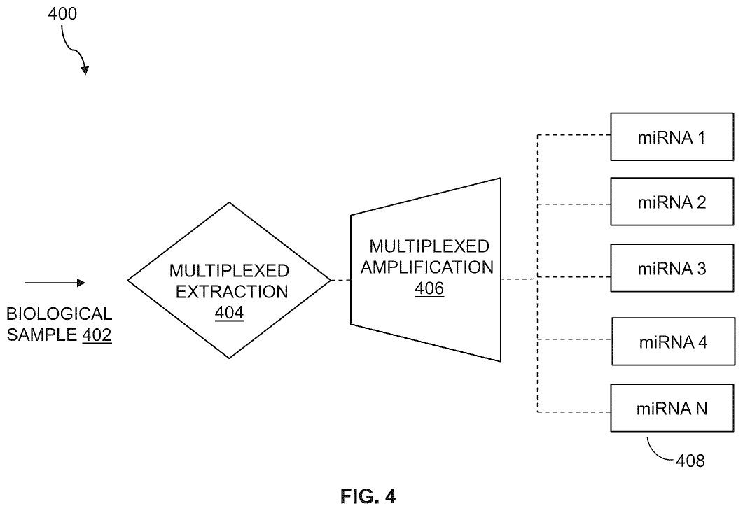

FIG. 4 illustrates an exemplary flow diagram representation of a method 400 for performing multiplexed extraction and nucleic acid amplification of miRNAs from the biological sample, according to an example embodiment of the present disclosure.

At step 402, the method 400 (e.g., the artificial intelligence based (AI-based) analyzing method) includes, receiving, by the artificial intelligence based (AI-based) analyzing system 102, the biological sample. At step 404, the method 400 includes multiplexed extraction, by the artificial intelligence based (AI-based) analyzing system 102, of the plurality of micro-ribonucleic acid (miRNA) concentrations from the one or more candidate pools of the plurality of miRNA concentrations in the biological sample. At step 406, the method 400 includes multiplexed amplification, using the plurality of primers, of the plurality of pre-defined nucleic acid sequences in the multiplexed extraction of the plurality of miRNA concentrations. At step 408, the method 400 includes identifying, by the artificial intelligence based (AI-based) analyzing system 102, the plurality of miRNA signature sequences and profiles indicative of the exposure to the plurality of biotic and abiotic agents, based on the miRNA profiling data.

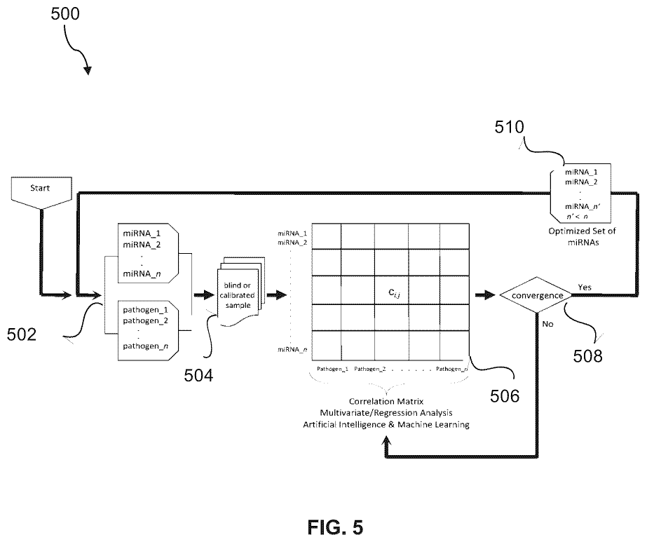

FIG. 5 illustrates an exemplary flow diagram representation of a method 500 (e.g., the artificial intelligence based (AI-based) analyzing method) for optimizing a set of miRNAs with a sample set of pathogens used in an earlier test for the response of the biotic and abiotic agents in a blind biological sample, according to an example embodiment of the present disclosure.

At step 502, the method 500 includes identifying, by the artificial intelligence based (AI-based) analyzing system 102, the plurality of miRNA signature and profiles sequences indicative of the exposure to the plurality of biotic and abiotic agents, based on the analyzed miRNA profiling data. The plurality of miRNA signature sequences and profiles is identified using the one or more statistical model-based techniques.

At step 504, the method 500 includes receiving, by the artificial intelligence based (AI-based) analyzing system 102, a blind/calibrated biological sample.

At step 506, the method 500 includes generating, by the artificial intelligence based (AI-based) analyzing system 102, the multidimensional correlation matrix corresponding to the plurality of miRNA signature sequences and profiles with the plurality of biotic and abiotic agents using the one or more statistical model-based techniques.

At step 508, the method 500 includes determining, by the artificial intelligence based (AI-based) analyzing system 102, a convergence of the plurality of miRNA signature sequences and profiles into the pathway corresponding to the plurality of biotic and abiotic agents, based on the generated multidimensional correlation matrix.

At step 510, the method 500 includes generating, by the artificial intelligence based (AI-based) analyzing system 102, the optimized subset from the plurality of miRNA signature sequences and profiles, when the first subset of the plurality of miRNA signature sequences and profiles is determined to be converged into the pathway. The artificial intelligence based (AI-based) analyzing system 102 further generates the second subset multidimensional correlation matrix corresponding to the second subset of the plurality of miRNA signature sequences and profiles, when the second subset of the plurality of miRNA signature sequences and profiles is determined to be not converged into the pathway. Further, the artificial intelligence based (AI-based) analyzing system 102 compares each of the first subset and the second subset of the plurality of miRNA signature sequences and profiles with each of the plurality of pre-defined miRNA signature sequences and profiles stored in the database 104. The artificial intelligence based (AI-based) analyzing system 102 evaluates the statistical confidence level of the multidimensional correlation matrix and the second subset multidimensional correlation matrix, based on the result of the comparison. The artificial intelligence based (AI-based) analyzing system 102 detects the response of the pathogen in the blind biological sample using the first subset of the plurality of miRNA signature sequences and profiles, based on the result of the evaluation.

For example, a maximum potential for multiplexity of extraction and amplification can be estimated as, for example, total number of miRNAs that might be related to pathogens is close to or more than 50,000 according to a miRBase database (v22) release and beyond. Consider, the total number of known and diagnostically useful miRNAs is variable ‘N,’ where the variable ‘N’ is a large number (>10). Taking N=50k as a figure of merit for the purpose of estimate, and assuming the noise floor for multiplexity is two miRNAs. Any group of two miRNAs or more could theoretically indicate biotic and abiotic response. Accordingly, a largest combinatorial space of multiplexity for miRNA base testing is P (N), which is expressed in equation 1 below:

$$P ( N ) = {\sum }_{m = 2}^{N} ( Nm ) = {\sum }_{m = 2}^{N} \frac{N !}{( N - m ) ! m !} \approx {2}^{N} \qquad Equation 1$$

For example, for N=50k, this number is practically unbound, opening the theoretical space for miRNA base detection to practically any number of known pathogens or diseases.

In an example, the present disclosure may be implemented in two steps: (1) miRNA discovery and calibration (2) application to blind samples. The first step is accomplished by collecting the sample. A sample can be processed by splitting it into the number of the first candidate pool of miRNAs then performing individual extraction and nucleic acid amplification or performing a multiplexed extraction and amplification of the pool of miRNAs, as shown in FIGS. 3 and 4. For multiplexed extraction and amplification and subsequent analysis, the biological sample is obtained and prepared using microfluidics associated with the artificial intelligence based (AI-based) analyzing system 102. The amplifying unit 114 includes a thermal cycler or isothermal unit (not shown) for controlling heating, cooling, or steady state temperatures. The components of the amplifying unit 114 used for amplification reaction are assembled by a fluidics module (not shown).

After amplification, analysis is performed on the extracted miRNA concentrations using models including but not limited to regression analysis, multivariate analysis, artificial intelligence, and machine learning to compare to a set of known diseases in the sample using a known assay. This process can be reiterated to optimize the number of miRNAs needed for convergence of the analysis with the desired statistical confidence level. This leads to an optimized subset of miRNAs for the next step. The second step is to use the optimized set of miRNAs with the sample set of pathogens used in the first step to test for the response of the pathogen in a blind sample and produce results, as shown in FIG. 5.

### Exemplary Scenario

Consider blood and muscle tissue samples collected from the subject such as a captive white-tailed deer. Blood samples may be obtained from the jugular vein or collected from blood pooling within the neck area and placed into collection vials. Skeletal muscle samples may be frozen, placed on ice, or preserved in RNA later. The data, including locality, date of collection, sex, tissue type, and sampling conditions, may be incorporated into the database 104. Consider, Obex and medial retropharyngeal lymph nodes collected from all 300 captive white-tailed deer. The collected 300 blood and muscle tissue samples from captive white-tailed deer may be used for research on Chronic Wasting Disease (CWD). Consider 93 samples for miRNA biomarker assays. Immunohistochemistry may be performed on lymph nodes and obex tissue to validate the diagnostic potential of the miRNA biomarker assays. The artificial intelligence based (AI-based) analyzing system 102 may extract total RNA from deer serum samples and achieve cDNA synthesis. Further, the system 102 may evaluate 5 miRNA primer sets to determine the diagnostic potential of miRNAs as biomarkers for detecting CWD prion production.

The artificial intelligence based (AI-based) analyzing system 102 may amplify cDNA and quantify using a green polymerase chain reaction kit. The artificial intelligence based (AI-based) analyzing system 102 may score serum quality using hemolysis detection methods, and exclusion criteria were established to evaluate the samples. The artificial intelligence based (AI-based) analyzing system 102 applies three criteria to assess anticipated variation in sample quality. The artificial intelligence based (AI-based) analyzing system 102 may divide samples into three subsets based on hemoglobin concentration and successful amplification of miRNA A. The artificial intelligence based (AI-based) analyzing system 102 may perform statistical and discriminant function analysis on the subsets. For example, the results may be visualized using scatterplot matrices, canonical plots, and logistic regression. The artificial intelligence based (AI-based) analyzing system 102 may use an independent approach to a discrimination function analyses (DFA) to assign threshold parameters for miRNA designation. This approach used pair-wise regression analyses of miRNAs coupled with a “visual” partitioning of the a priori scoring of individual samples by a pre-determined laboratory to determine correlations and diagnostic capabilities of miRNAs. The study designated upper and lower thresholds for miRNAs in each pair-wise comparison.

In an example, the artificial intelligence based (AI-based) analyzing system 102 may identify three putative diagnostic miRNAs (C, D, and E) for prion infection in animals with the CWD. A total of 28 samples may be excluded from downstream analyses due to excessive hemolysis and poor amplification of miRNA A. The remaining 71 samples may be divided, by the artificial intelligence based (AI-based) analyzing system 102, into three groups based on serum degradation. The artificial intelligence based (AI-based) analyzing system 102 assesses the influence of each putative diagnostic miRNA using a discriminant function analysis. The artificial intelligence based (AI-based) analyzing system 102 determines a distribution of calculated relative abundance “ΔCt” values, and pair-wise regression analyses is used to assign threshold parameters for miRNA designation. In an example, the artificial intelligence based (AI-based) analyzing system 102 may apply quantitative discriminant function analysis (QDFA) to the qPCR results for three putative diagnostic miRNAs to determine the degree of separation among groups of positive and negative individuals and to assess congruence between the miRNA biomarker test and diagnoses by the IHC. The scatterplot matrices of QDFA may provide predictive values of all three markers that collectively might constitute a valid miRNA assay for overall classification of specimens from CWD positive and negative animals. The miRNA assay classifies CWD positive and negative animals with 93.9% accuracy overall in Group I, 84.6% accuracy overall in Group II, and 59.2% accuracy overall in Group III. The sensitivity and specificity of the miRNA assay may be varied among the different groups.

Further, the artificial intelligence based (AI-based) analyzing system 102 uses a hierarchical diagnostic protocol with miRNA C as the most strongly discriminating component, and collectively applying data from all three diagnostic markers may produce an optimal, miRNA-based, high throughput assay for CWD infection. The accuracy of the assay may be decreased with sample degradation, and only a minimum subset of samples met the criteria to generate accurate data. Further, the miRNA C may have strongest degree of association with CWD in samples with minimal serum degradation and contributed the greatest weight in determining classification between positive and negative groups. Comparatively, miRNA D and E may be less informative.

Further, below table 1 shows the results of a discriminant function analysis of three miRNA markers in three data subsets. The data subsets may be classified based on ACt values, with positive or negative categories assigned based on immunohistochemistry (IHC) assays. Two of the seven IHC positive animals in Group I may only be positive in the retropharyngeal lymph node, indicating that pathological prion build-up had not yet reached the nervous system.

TABLE 1

| | | | DFA Reclassification | |
|---|---|---|---|---|
| | Classification by | | by miRNA assay | |
| Dataset | IHC (a priori) | | Negative | Positive |
| Group I, | Negative | (n = 26) | 24 | 2 |
| n = 33 | Positive | (n = 7) | 0 | 7 |
| Group II, | Negative | (n = 30) | 24 | 6 |
| n = 39 | Positive | (n = 9) | 0 | 9 |
| Group III, | Negative | (n = 54) | 28 | 26 |
| n = 71 | Positive | (n = 17) | 3 | 14 |

Further, below table 2 shows the effect of adjusting the upper and lower threshold of the miRNA assay on accuracy, sensitivity, and false positives. The results show that constraining the minimal diagnostic window decreased false positives, but at the expense of overall accuracy. The adjustments also affected sensitivity differently, with some markers showing modestly increased accuracy but at the expense of sensitivity. The table also indicates that the three miRNA markers were not redundantly informative, as adjusting the capture window required alteration of different miRNAs' partitions.

TABLE 2

| Constrained | | ΔCt | | | |
|---|---|---|---|---|---|
| sensitivity | | partition | | False | Accuracy |
| (%) | miRNA | (cycles) | Comparison | positives | (%) |
| 100% | C | 4.00 | C vs E | 8/15 = 53.3% | 75.8% |
| | D | 8.75 | C vs D | 6/13 = 46.2% | 81.8% |
| | E | 10.00 | D vs E | 14/21 = 66.7% | 57.6% |
| 75% | C | 3.75 | C vs E | 2/6 = 33.3% | 84.8% |
| | D | 7.5 | C vs D | 2/6 = 33.3% | 84.8% |
| | E | 8.75 | D vs E | 5/9 = 50.0% | 75.8% |
| 50% | C | 3.75 | C vs E | 1/4 = 25.0% | 84.8% |
| | D | 7.5 | C vs D | 1/4 = 25.0% | 84.8% |
| | E | 8.25 | D vs E | 2/5 = 40.0% | 81.8% |

Furthermore, below table 3 shows the congruence of different methods for detecting chronic wasting disease (CWD) infection, including immunohistochemistry (IHC), enzyme-linked immunosorbent assay (ELISA), protein misfolding cyclic amplification (PMCA), real-time quaking-induced conversion (RT-QuIC), Minnesota quaking-induced conversion (MN-QuiC), and the miRNA assay developed in this study. The table shows the inter-assay accuracy calculations, which reflect the degree of agreement between the different methods. The miRNA assay showed moderate agreement with the other methods, with the highest agreement observed between the Real-Time Quaking-Induced Conversion (RT-QuIC) and MN-QuiC assays.

TABLE 3

| | ELISA | PMCA | RT-QuIC | MN-QuIC | miRNA |
|---|---|---|---|---|---|
| IHC | 78% | 89% | 83% | 100% | 94% |
| ELISA | | 90% | 92% | 100% | N/A |
| PMCA | | | 80% | N/A | N/A |
| RT-QuIC | | | | 100% | N/A |
| MN-QuIC | | | | | N/A |

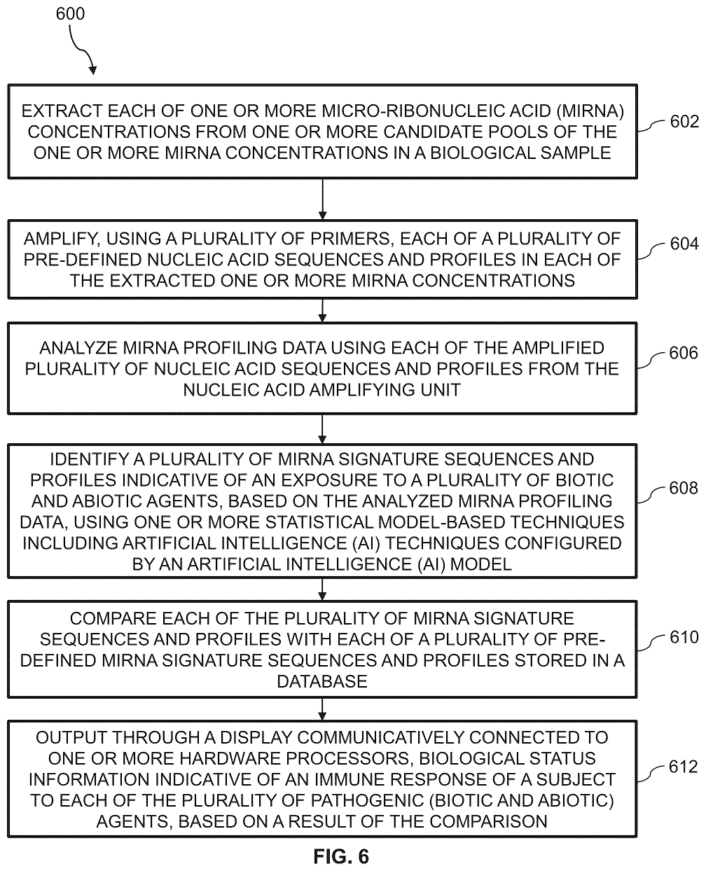

FIG. 6 illustrates a flow chart depicting the artificial intelligence based (AI-based) analyzing method 600 for analyzing the micro-ribonucleic acid (miRNA) signature sequences and profiles in the biological sample to identify the response of the subject to the biotic and abiotic agents, according to an example embodiment of the present disclosure.

At block 602, the artificial intelligence based (AI-based) analyzing method 600 may include extracting, by the one or more hardware processors 114 through the extractor unit 110, each of the one or more micro-ribonucleic acid (miRNA) concentrations from the one or more candidate pools of the one or more miRNA concentrations in the biological sample.

At block 604, the artificial intelligence based (AI-based) analyzing method 600 may include amplifying, by the one or more hardware processors 114 through the nucleic acid amplifying unit 112, using the plurality of primers, each of the plurality of pre-defined nucleic acid sequences and profiles in each of the extracted the one or more miRNA concentrations.

At block 606, the artificial intelligence based (AI-based) analyzing method 600 may include analyzing, by the one or more hardware processors 114, the miRNA profiling data using each of the amplified plurality of nucleic acid sequences and profiles from the nucleic acid amplifying unit 112.

At block 608, the artificial intelligence based (AI-based) analyzing method 600 may include identifying, by the one or more hardware processors 114, the plurality of miRNA signature sequences and profiles indicative of the exposure to the plurality of biotic and abiotic agents, based on the analyzed miRNA profiling data. In an embodiment, the plurality of miRNA signature sequences is identified using one or more statistical model-based techniques. In another embodiment, the one or more statistical model-based techniques comprise artificial intelligence (AI) techniques configured by the artificial intelligence (AI) model.

At block 610, the artificial intelligence based (AI-based) analyzing method 600 may include comparing, by the one or more hardware processors 114, each of the plurality of miRNA signature sequences and profiles with each of the plurality of pre-defined miRNA signature sequences and profiles stored in the database 104.

At block 612, the artificial intelligence based (AI-based) analyzing method 600 may include outputting, by the one or more hardware processors 114, through the display communicatively connected to the one or more hardware processors 114, the biological status information indicative of the response of the subject to each of the plurality of biotic and abiotic agents, based on the result of the comparison.

The artificial intelligence based (AI-based) analyzing method 600 may be implemented in any suitable hardware, software, firmware, or combination thereof. The order in which the artificial intelligence based (AI-based) analyzing method 600 is described is not intended to be construed as a limitation, and any number of the described method blocks may be combined or otherwise performed in any order to implement the artificial intelligence based (AI-based) analyzing method 600 or an alternate method. Additionally, individual blocks may be deleted from the artificial intelligence based (AI-based) analyzing method 600 without departing from the spirit and scope of the present disclosure described herein. Furthermore, the artificial intelligence based (AI-based) analyzing method 600 may be implemented in any suitable hardware, software, firmware, or a combination thereof, that exists in the related art or that is later developed. The artificial intelligence based (AI-based) analyzing method 600 describes, without limitation, the implementation of the artificial intelligence based (AI-based) analyzing system 102. A person of skill in the art will understand that artificial intelligence based (AI-based) analyzing method 600 may be modified appropriately for implementation in various manners without departing from the scope and spirit of the disclosure.

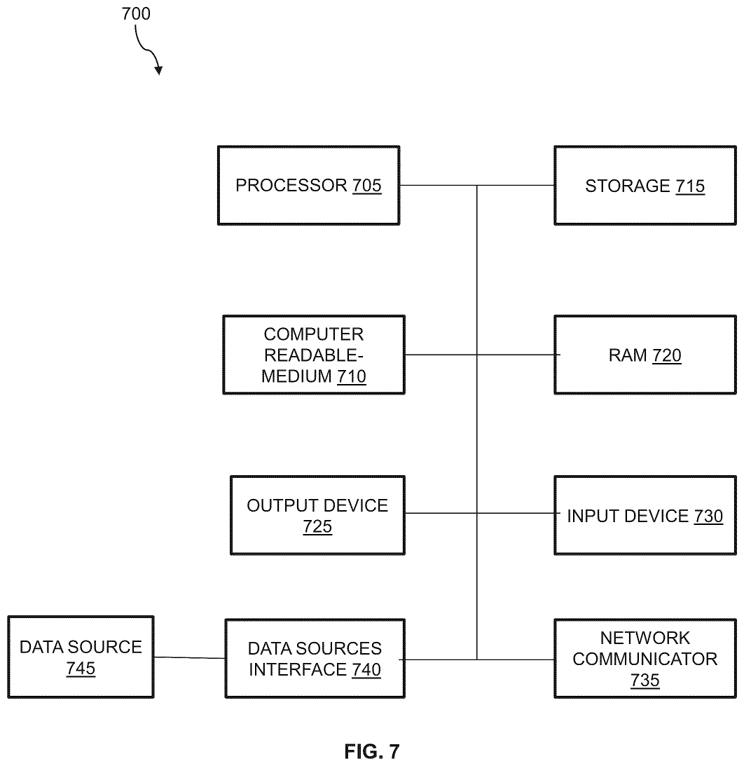

FIG. 7 illustrates an exemplary block diagram representation of a hardware platform 700 for implementation of the disclosed artificial intelligence based (AI-based) analyzing system 102, according to an example embodiment of the present disclosure. For the sake of brevity, the construction, and operational features of the artificial intelligence based (AI-based) analyzing system 102 which are explained in detail above are not explained in detail herein. Particularly, computing machines such as but not limited to internal/external server clusters, quantum computers, desktops, laptops, smartphones, tablets, and wearables which may be used to execute the artificial intelligence based (AI-based) analyzing system 102 or may include the structure of the hardware platform 700. As illustrated, the hardware platform 700 may include additional components not shown, and some of the components described may be removed and/or modified. For example, a computer system with multiple GPUs may be located on external-cloud platforms including Amazon Web Services, or internal corporate cloud computing clusters, or organizational computing resources.

The hardware platform 700 may be a computer system such as the system 102 (i.e., the artificial intelligence based (AI-based) analyzing system 102) that may be used with the embodiments described herein. The computer system may represent a computational platform that includes components that may be in a server or another computer system. The computer system may execute, by the processor 705 (e.g., single, or multiple processors) or other hardware processing circuits, the methods, functions, and other processes described herein. These methods, functions, and other processes may be embodied as machine-readable instructions stored on a computer-readable medium, which may be non-transitory, such as hardware storage devices (e.g., RAM (random access memory), ROM (read-only memory), EPROM (erasable, programmable ROM), EEPROM (electrically erasable, programmable ROM), hard drives, and flash memory). The computer system may include the processor 705 that executes software instructions or code stored on a non-transitory computer-readable storage medium 710 to perform methods of the present disclosure. The software code includes, for example, instructions to gather data and analyze the data. For example, the plurality of modules 118 includes the profile analyzing module 206, the signature identifying module 208, the database comparing module 210, the result outputting module 212, the correlation generating module 214, the converge determining module 216, the optimized set generating module 218, the level evaluating module 220, the pathogen detecting module 222, and the training module 224.

The instructions on the computer-readable storage medium 710 are read and stored the instructions in storage 715 or random-access memory (RAM). The storage 715 may provide a space for keeping static data where at least some instructions could be stored for later execution. The stored instructions may be further compiled to generate other representations of the instructions and dynamically stored in the RAM such as RAM 720. The processor 705 may read instructions from the RAM 720 and perform actions as instructed.

The computer system may further include the output device 725 to provide at least some of the results of the execution as output including, but not limited to, visual information to users, such as external agents. The output device 725 may include a display on computing devices and virtual reality glasses. For example, the display may be a mobile phone screen or a laptop screen. GUIs and/or text may be presented as an output on the display screen. The computer system may further include an input device 730 to provide a user or another device with mechanisms for entering data and/or otherwise interacting with the computer system. The input device 730 may include, for example, a keyboard, a keypad, a mouse, or a touchscreen. Each of these output devices 725 and input device 730 may be joined by one or more additional peripherals. For example, the output device 725 may be used to display the results such as bot responses by the executable chatbot.

A network communicator 735 may be provided to connect the computer system to a network and in turn to other devices connected to the network including other clients, servers, data stores, and interfaces, for example. A network communicator 735 may include, for example, a network adapter such as a LAN adapter or a wireless adapter. The computer system may include a data sources interface 740 to access the data source 745. The data source 745 may be an information resource. As an example, a database of exceptions and rules may be provided as the data source 745. Moreover, knowledge repositories and curated data may be other examples of the data source 745.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limited, of the scope of the invention, which is outlined in the following claims.

What is claimed is:

1. An artificial intelligence based (AI-based) analyzing system for analyzing micro-ribonucleic acid (miRNA) signature sequences and profiles in a biological sample, the artificial-intelligence based (AI-based) analyzing system comprising:
an extractor unit configured to extract each of one or more micro-ribonucleic acid (miRNA) concentrations from one or more candidate pools of the one or more miRNA concentrations in the biological sample;
a nucleic acid amplifying unit, communicatively connected to the extractor unit, configured to amplify, using a plurality of primers, each of a plurality of pre-defined nucleic acid sequences and profiles in each of the extracted one or more miRNA concentrations; and
one or more hardware processors communicatively connected to the nucleic acid amplifying unit; and a memory coupled to the one or more hardware processors, wherein the memory comprises a plurality of modules in form of programmable instructions executable by the one or more hardware processors, wherein the plurality of modules comprises:
a profile analyzing module configured to analyze miRNA profiling data using each of the amplified plurality of nucleic acid sequences and profiles from the nucleic acid amplifying unit;
a signature identifying module configured to identify a plurality of miRNA signature sequences and profiles indicative of an exposure to a plurality of biotic and abiotic agents cause change, based on the analyzed miRNA profiling data, wherein the plurality of miRNA signature sequences and profiles is identified using one or more statistical model-based techniques, and wherein the one or more statistical model-based techniques comprise artificial intelligence (AI) techniques configured by an artificial intelligence (AI) model;
a database comparing module configured to compare each of the plurality of miRNA signature sequences and profiles with each of a plurality of pre-defined miRNA signature sequences and profiles stored in a database; and
a result outputting module configured to output, through a display communicatively connected to the one or more hardware processors, biological status information indicative of a response of a subject to each of the plurality of biotic and abiotic agents, based on a result of the comparison,
wherein the plurality of biotic and abiotic agents comprises at least one of: one or more biotic organisms and abiotic stimuli, one or more pathogenesis-related (PR) proteins, one or more antimicrobial peptides (AMPs) induced by phytopathogens, unregulated tissue growth including benign and cancerous tumors, and circulating tumor cells, wherein the one or more biotic organisms comprise at least one of: bacteria, fungi, protozoa, worms, viruses, and parasites, and wherein the one or more pathogenesis-related (PR) proteins comprise prions.

2. The artificial intelligence based (AI-based) analyzing system of claim 1, wherein the plurality of modules further comprises:
a correlation generating module configured to generate a multidimensional correlation matrix corresponding to the plurality of miRNA signature sequences and profiles with the plurality of biotic and abiotic agents using the one or more statistical model-based techniques;
a converge determining module configured to determine a convergence of the plurality of miRNA signature sequences and profiles into a pathway corresponding to the plurality of biotic and abiotic agents, based on the generated multidimensional correlation matrix;
an optimized set generating module configured to generate an optimized subset from the plurality of miRNA signature sequences and profiles, when a first subset of the plurality of miRNA signature sequences and profiles is determined to be converged into the pathway;
the correlation generating module configured to generate a second subset multidimensional correlation matrix corresponding to a second subset of the plurality of miRNA signature sequences and profiles, when a second subset of the plurality of miRNA signature sequences and profiles is determined to be not converged into the pathway;
the database comparing module configured to compare each of the first subset and the second subset of the plurality of miRNA signature sequences and profiles with each of a plurality of pre-defined miRNA signature sequences and profiles stored in the database;
a level evaluating module configured to evaluate a statistical confidence level of the multidimensional correlation matrix and the second subset multidimensional correlation matrix, based on a result of the comparison; and
a biotic and abiotic agent detecting module configured to detect a response in a blind biological sample using the first subset of the plurality of miRNA signature sequences and profiles, based on a result of the evaluation.

3. The artificial intelligence based (AI-based) analyzing system of claim 1, wherein to identify the plurality of miRNA signature sequences and profiles, the signature identifying module is further configured to:
determine at least one of:
one or more differentially expressed miRNA concentrations corresponding to at least one of biological contexts and biological conditions, using the one or more statistical model-based techniques, and
one or more miRNA expression patterns associated with at least one of biological processes and disease states, using the one or more statistical model-based techniques.

4. The artificial intelligence based (AI-based) analyzing system of claim 1, wherein amplifying each of the plurality of pre-defined nucleic acid sequences is based on at least one of thermal-cyclic polymerase enzymes and isothermal polymerase enzymes.

5. The artificial intelligence based (AI-based) analyzing system of claim 1, wherein in identifying, by the artificial intelligence (AI) model, the plurality of miRNA signature sequences and profiles, based on the analyzed miRNA profiling data, the signature identifying module is configured to:
obtain the miRNA profiling data from the profile analyzing module;
process the miRNA profiling data by at least one of: removing one or more outliers associated with the miRNA profiling data, normalizing one or more expression values associated with the miRNA profiling data, and handling one or more missing values associated with the miRNA profiling data;
extract one or more features from the miRNA profiling data, wherein the extracted one or more features comprise at least one of: one or more expression levels of the miRNAs, one or more statistical measures comprising mean and standard values, and one or more information comprising at least one of: target gene information and pathway enrichment, derived from one or more biological insights; and
identify the plurality of miRNA signature sequences and profiles indicative of the exposure to the plurality of biotic and abiotic agents cause change, based on feature space associated with the miRNA profiling data, using the artificial intelligence (AI) model,
wherein the miRNA profiling data comprises at least one of a miRNA response, a miRNA absence, miRNA quantitative levels, miRNA concentrations, miRNA expression patterns, and miRNA relationships within the miRNA profiling data.

6. The artificial intelligence based (AI-based) analyzing system of claim 5, further comprising a training module configured to train the artificial intelligence (AI) model, wherein in training the artificial intelligence (AI) model, the training module is configured to:
obtain one or more training datasets associated with the analyzed miRNA profiling data;
train the artificial intelligence (AI) model on the one or more training datasets to identify the plurality of miRNA signature sequences and profiles, wherein the one or more training datasets comprise at least one of: one or more proprietary data generated in a workflow and one or more information corresponding to the analyzed miRNA profiling data;
validate performance of the artificial intelligence (AI) model on one or more validation datasets;
update one or more hyperparameters associated with the analyzed miRNA profiling data to optimize the performance of the artificial intelligence (AI) model in identifying the plurality of miRNA signature sequences and profiles; and
evaluate the trained artificial intelligence (AI) model using one or more metrics comprising at least one of: accuracy metric, precision metric, recall metric, F1-score metric, Area Under the Precision Recall Curve (AUPRC), Receiver operating characteristic (ROC) curves, and Multi-Class Confusion Matrices.

7. The artificial intelligence based (AI-based) analyzing system of claim 1, further comprises:
the extractor unit configured for multiplexed extraction of the plurality of micro-ribonucleic acid (miRNA) concentrations from the one or more candidate pools of the plurality of miRNA concentrations in the biological sample;
the nucleic acid amplifying unit configured for multiplexed amplification of the plurality of pre-defined nucleic acid sequences in the multiplexed extraction of the plurality of miRNA concentrations, using the plurality of primers;
the profile analyzing module configured to analyze the miRNA profiling data using the multiplexed amplification of the plurality of nucleic acid sequences and profiles from the nucleic acid amplifying unit;
the signature identifying module configured to identify the plurality of miRNA signature sequences and profiles indicative of the exposure to the plurality of biotic and abiotic agents, based on the analyzed miRNA profiling data, wherein the plurality of miRNA signature sequences and profiles is identified using the one or more statistical model based techniques;
the database comparing module configured to compare each of the plurality of miRNA signature sequences and profiles with each of the plurality of pre-defined miRNA signature sequences and profiles stored in the database; and
the result outputting module configured to output, through the display communicatively connected to the one or more hardware processors, the biological status information indicative of the response of the subject to each of the plurality of biotic and abiotic agents, based on the result of the comparison.

8. The artificial intelligence based (AI-based) analyzing system of claim 1, wherein the miRNA profiling data comprises at least one of a miRNA response, a miRNA absence, miRNA quantitative levels, miRNA concentrations, miRNA expression patterns, and miRNA relationships within the miRNA profiling data.

9. An artificial intelligence based (AI-based) analyzing method for analyzing micro-ribonucleic acid (miRNA) signature sequences and profiles in a biological sample, the artificial intelligence based (AI-based) analyzing method comprising:
extracting, by one or more hardware processors through an extractor unit, each of one or more micro-ribonucleic acid (miRNA) concentrations from one or more candidate pools of the one or more miRNA concentrations in a biological sample;
amplifying, by the one or more hardware processors through a nucleic acid amplifying unit, using a plurality of primers, each of a plurality of pre-defined nucleic acid sequences and profiles in each of the extracted one or more miRNA concentrations;
analyzing, by the one or more hardware processors, miRNA profiling data using each of the amplified plurality of nucleic acid sequences and profiles from the nucleic acid amplifying unit;
identifying, by the one or more hardware processors, a plurality of miRNA signature sequences and profiles indicative of an exposure to a plurality of biotic and abiotic agents cause change, based on the analyzed miRNA profiling data, wherein the plurality of miRNA signature sequences and profiles is identified using one or more statistical model-based techniques, and wherein the one or more statistical model-based techniques comprise artificial intelligence (AI) techniques configured by an artificial intelligence (AI) model;
comparing, by the one or more hardware processors, each of the plurality of miRNA signature sequences and profiles with each of a plurality of pre-defined miRNA signature sequences and profiles stored in a database; and
outputting, by the one or more hardware processors, through a display communicatively connected to the one or more hardware processors, biological status information indicative of a response of a subject to each of the plurality of biotic and abiotic agents, based on a result of the comparison,
wherein the plurality of biotic and abiotic agents comprises at least one of: one or more biotic organisms and abiotic stimuli, one or more pathogenesis-related (PR) proteins, one or more antimicrobial peptides (AMPs) induced by phytopathogens, unregulated tissue growth including benign and cancerous tumors, and circulating tumor cells, wherein the one or more biotic organisms comprise at least one of: bacteria, fungi, protozoa, worms, viruses, and parasites, and wherein the one or more pathogenesis-related (PR) proteins comprise prions.

10. The artificial intelligence based (AI-based) analyzing method of claim 9, further comprising:
generating, by the one or more hardware processors, a multidimensional correlation matrix corresponding to the plurality of miRNA signature sequences and profiles with the plurality of biotic and abiotic agents using the one or more statistical model-based techniques;
determining, by the one or more hardware processors, a convergence of the plurality of miRNA signature sequences and profiles into a pathway corresponding to the plurality of biotic and abiotic agents, based on the generated multidimensional correlation matrix;
generating, by the one or more hardware processors, an optimized subset from the plurality of miRNA signature sequences and profiles, when a first subset of the plurality of miRNA signature sequences and profiles is determined to be converged into the pathway;
generating, by the one or more hardware processors, a second subset multidimensional correlation matrix corresponding to a second subset of the plurality of miRNA signature sequences and profiles, when a second subset of the plurality of miRNA signature sequences and profiles is determined to be not converged into the pathway;
comparing, by the one or more hardware processors, each of the first subset and the second subset of the plurality of miRNA signature sequences and profiles with each of a plurality of pre-defined miRNA signature sequences and profiles stored in the database;
evaluating, by the one or more hardware processors, a statistical confidence level of the multidimensional correlation matrix and the second subset multidimensional correlation matrix, based on a result of the comparison; and
detecting, by the one or more hardware processors, a response of a pathogen in a blind biological sample using the first subset of the plurality of miRNA signature sequences and profiles, based on a result of the evaluation.

11. The artificial intelligence based (AI-based) analyzing method of claim 9, wherein identifying the plurality of miRNA signature sequences and profiles further comprises:
determining, by the one or more hardware processors, at least one of:
one or more differentially expressed miRNA concentrations corresponding to at least one of biological contexts and biological conditions, using the one or more statistical model-based techniques, and
one or more miRNA expression patterns associated with at least one of biological processes and disease states, using the one or more statistical model-based techniques.

12. The artificial intelligence based (AI-based) analyzing method of claim 9, wherein amplifying each of the plurality of pre-defined nucleic acid sequences is based on at least one of thermal-cyclic polymerase enzymes and isothermal polymerase enzymes.

13. The artificial intelligence based (AI-based) analyzing method of claim 9, wherein identifying, by the artificial intelligence (AI) model, the plurality of miRNA signature sequences and profiles, based on the analyzed miRNA profiling data, comprises:
obtaining, by the one or more hardware processors, the miRNA profiling data from the profile analyzing module;
processing, by the one or more hardware processors, the miRNA profiling data by at least one of: removing one or more outliers associated with the miRNA profiling data, normalizing one or more expression values associated with the miRNA profiling data, and handling one or more missing values associated with the miRNA profiling data;
extracting, by the one or more hardware processors, one or more features from the miRNA profiling data, wherein the extracted one or more features comprise at least one of: one or more expression levels of the miRNAs, one or more statistical measures comprising mean and standard values, and one or more information comprising at least one of: target gene information and pathway enrichment, derived from one or more biological insights; and
identifying, by the one or more hardware processors, the plurality of miRNA signature sequences and profiles indicative of the exposure to the plurality of biotic and abiotic agents cause change, based on feature space associated with the miRNA profiling data, using the artificial intelligence (AI) model.

14. The artificial intelligence based (AI-based) analyzing method of claim 13, further comprising training, by the one or more hardware processors, the artificial intelligence (AI) model, wherein training the artificial intelligence (AI) model comprises:
obtaining, by the one or more hardware processors, one or more training datasets associated with the analyzed miRNA profiling data;
training, by the one or more hardware processors, the artificial intelligence (AI) model on the one or more training sets to identify the plurality of miRNA signature sequences and profiles, wherein the one or more training datasets comprise at least one of: one or more proprietary data generated in a workflow and one or more information corresponding to the analyzed miRNA profiling data;
validating, by the one or more hardware processors, performance of the artificial intelligence (AI) model on one or more validation datasets;
updating, by the one or more hardware processors, one or more hyperparameters associated with the analyzed miRNA profiling data to optimize the performance of the artificial intelligence (AI) model in identifying the plurality of miRNA signature sequences and profiles; and
evaluating, by the one or more hardware processors, the trained artificial intelligence (AI) model using one or more metrics comprising at least one of: accuracy metric, precision metric, recall metric, F1-score metric, Area Under the Precision Recall Curve (AUPRC), Receiver operating characteristic (ROC) curves, and Multi-Class Confusion Matrices.

15. The artificial intelligence based (AI-based) analyzing method of claim 9, wherein the miRNA profiling data comprises at least one of a miRNA response, a miRNA absence, miRNA quantitative levels, miRNA concentrations, miRNA expression patterns, and miRNA relationships within the miRNA profiling data.

16. The artificial intelligence based (AI-based) analyzing method of claim 9, further comprising:
multiplexed extraction, by the one or more hardware processors through the extractor unit, of the plurality of micro-ribonucleic acid (miRNA) concentrations from the one or more candidate pools of the plurality of miRNA concentrations in the biological sample;
multiplexed amplification, by the one or more hardware processors through the nucleic acid amplifying unit, of the plurality of pre-defined nucleic acid sequences in the multiplexed extraction of the plurality of miRNA concentrations, using the plurality of primers;
analyzing, by the one or more hardware processors, the miRNA profiling data using the multiplexed amplification of the plurality of nucleic acid sequences and profiles from the nucleic acid amplifying unit;
identifying, by the one or more hardware processors, the plurality of miRNA signature sequences and profiles indicative of the exposure to the plurality of biotic and abiotic agents, based on the analyzed miRNA profiling data, wherein the plurality of miRNA signature sequences and profiles is identified using the one or more statistical model-based techniques;
comparing, by the one or more hardware processors, each of the plurality of miRNA signature sequences and profiles with each of the plurality of pre-defined miRNA signature sequences stored in the database; and
outputting, by the one or more hardware processors, through the display communicatively connected to the one or more hardware processors, the biological status information indicative of the response of a subject to each of the plurality of biotic and abiotic agents, based on the result of the comparison.

17. The artificial intelligence based (AI-based) analyzing method of claim 16, wherein the multiplexed amplification of the plurality of pre-defined nucleic acid sequences is based on at least one of the thermal-cyclic polymerase enzymes and the isothermal polymerase enzymes.

18. A non-transitory computer-readable storage medium having programmable instructions stored therein, that when executed by one or more hardware processors, cause the one or more hardware processors to:
analyze miRNA profiling data using each of an amplified plurality of nucleic acid sequences and profiles from a nucleic acid amplifying unit; and
identify a plurality of miRNA signature sequences and profiles indicative of an exposure to a plurality of biotic and abiotic agents cause change, based on the analyzed miRNA profiling data, wherein the plurality of miRNA signature sequences and profiles is identified using one or more statistical model-based techniques, and wherein the one or more statistical model-based techniques comprise artificial intelligence (AI) techniques configured by an artificial intelligence (AI) model.
compare each of the plurality of miRNA signature sequences and profiles with each of a plurality of pre-defined miRNA signature sequences and profiles stored in a database; and
output, through a display communicatively connected to the one or more hardware processors, biological status information indicative of a response of a subject to each of the plurality of biotic and abiotic agents, based on a result of the comparison.

19. The non-transitory computer-readable storage medium of claim 18, wherein the one or more hardware processors are further configured to:
generate a multidimensional correlation matrix corresponding to the plurality of miRNA signature sequences and profiles with the plurality of biotic and abiotic agents using the one or more statistical model-based techniques;
determine a convergence of the plurality of miRNA signature sequences and profiles into a pathway corresponding to the plurality of biotic and abiotic agents, based on the generated multidimensional correlation matrix;
generate an optimized subset from the plurality of miRNA signature sequences and profiles, when a first subset of the plurality of miRNA signature sequences and profiles is determined to be converged into the pathway;
generate a second subset multidimensional correlation matrix corresponding to a second subset of the plurality of miRNA signature sequences and profiles, when a second subset of the plurality of miRNA signature sequences and profiles is determined to be not converged into the pathway;
compare each of the first subset and the second subset of the plurality of miRNA signature sequences and profiles with each of a plurality of pre-defined miRNA signature sequences and profiles stored in the database;
evaluate a statistical confidence level of the multidimensional correlation matrix and the second subset multidimensional correlation matrix, based on a result of the comparison; and
detect a response to the biotic and abiotic agents in a blind biological sample using the first subset of the plurality of miRNA signature sequences and profiles, based on a result of the evaluation.

20. The non-transitory computer-readable storage medium of claim 18, wherein the one or more hardware processors are further configured to:
determine at least one of:
one or more differentially expressed miRNA concentrations corresponding to at least one of biological contexts and biological conditions, using the one or more statistical model-based techniques, and
one or more miRNA expression patterns associated with at least one of biological processes and disease states, using the one or more statistical model-based techniques.
